SEQUENCE LISTING (10 sequences)

SEQ ID NO: 1 - DNA, 167 bp, synthetic construct
  source 1..167: mol_type = other DNA
ttctgtgtcttcccacctacagtaacaaaggcatggagcatctgtacagcatgaagtgcaagaacgtggtgcccctctgtgacctgctgctggagatgctggacgcccaccgcctacatgcgcccactagccgtggaggggcatccgtggaggagacggaccaaagc

SEQ ID NO: 2 - DNA, 167 bp, synthetic construct
  source 1..167: mol_type = other DNA
ttctgtgtcttcccacctacagtaacaaaggcatggagcatctgtacagcatgaagtgcaagaacgtggtgcacctctatgacctgctgctggagatgctggacgcccaccgcctacatgcgcccactagccgtggaggggcatccgtggaggagacggaccaaagc

SEQ ID NO: 3 - DNA, 170 bp, synthetic construct
  source 1..170: mol_type = other DNA
ctggtcttgatttttcgtatatttcaggagataagttttaaaagtaagccgaatgtctggggatatttctcagttaaggtaagctgttcatagctctgttgcagtgtcatgttgttttgagttctgcagaaaattggtctgcaagattttttatttgaattagtattctc

SEQ ID NO: 4 - DNA, 65 bp, synthetic construct
  source 1..65: mol_type = other DNA
  modified_base 1: mod_base = OTHER; note = isocytosine
  misc_feature 33^34: note = C3 spacer
  misc_feature >65: note = C3 spacer
  modified_base 49: mod_base = OTHER; note = sugar is ribose
caaataaatatataataataactgacaccaaaaaaaacctgtcagttaaggtaagctgttcatag

SEQ ID NO: 5 - DNA, 57 bp, synthetic construct
  source 1..57: mol_type = other DNA
  misc_feature 32^33: note = C3 spacer
  modified_base 49: mod_base = OTHER; note = sugar is ribose
  misc_feature >57: note = C3 spacer
  modified_base 1: mod_base = OTHER; note = isocytosine
ctcctcttcttcttctttcagaggtaccaaaaaaaaacctacctctgtgacctgctg

SEQ ID NO: 6 - DNA, 55 bp, synthetic construct
  source 1..55: mol_type = other DNA
  misc_feature 32^33: note = C3 spacer
  modified_base 45: mod_base = OTHER; note = sugar is ribose
  misc_feature >55: note = C3 spacer
  modified_base 1: mod_base = OTHER; note = isocytosine
cccctcctcctccttcctccctgcacaccaaaaaacctgtgcacctctatgacct

SEQ ID NO: 7 - DNA, 17 bp, synthetic construct
  source 1..17: mol_type = other DNA
tgggcgtccagcatctc

SEQ ID NO: 8 - DNA, 21 bp, synthetic construct
  source 1..21: mol_type = other DNA
caaaggcatggagcatctgta

SEQ ID NO: 9 - DNA, 22 bp, synthetic construct
  source 1..22: mol_type = other DNA
aacaacatgacactgcaacaga

SEQ ID NO: 10 - DNA, 24 bp, synthetic construct
  source 1..24: mol_type = other DNA
aagccgaatgtctggggatatttc

(19) United States
(12) Patent Application Publication
(10) Pub. No.: US 2023/0068047 A1
(43) Pub. Date: Mar. 2, 2023

(54) DIGITAL AMPLIFICATION ASSAY ANALYSIS METHOD

(71) Applicant: LUMINEX CORPORATION, Austin, TX (US)

(72) Inventors: Jenni BERNIER, Austin, TX (US); Doug WHITMAN, Austin, TX (US)

(73) Assignee: LUMINEX CORPORATION, Austin, TX (US)

(21) Appl. No.: 17/820,277

(22) Filed: Aug. 17, 2022

Related U.S. Application Data
(60) Provisional application No. 63/234,850, filed on Aug. 19, 2021.

(51) Int. Cl. C12Q 1/6851; C12Q 1/686

(52) U.S. Cl. CPC C12Q 1/6851; C12Q 1/686

(57) ABSTRACT

Provided herein are methods and systems for detecting the presence or absence of multiple target nucleic acids in partitions of a digital amplification assay. In one embodiment of the method, different probes labeled with the same signal-generating label are distinguishable from each other as a result of having different melting temperatures in the presence of their cognate target nucleic acids. Following amplification of the target nucleic acids in a digital assay, signals are measured at three or more different temperatures and at least two relational values between signals measured at the three or more different temperatures are calculated and plotted against each other to classify partition subsets according to their target nucleic acid occupancy.

### CROSS-REFERENCE TO RELATED APPLICATIONS

This application claims priority to U.S. Provisional Patent Application Ser. No. 63/234,850, filed Aug. 19, 2021, the entire contents of which are incorporated herein by reference.

### REFERENCE TO A SEQUENCE LISTING

This application contains a Sequence Listing XML, which has been submitted electronically and is hereby incorporated by reference in its entirety. Said Sequence Listing XML, created on Sep. 19, 2022, is named LUMNP0156US_ST26_updated.xml and is 15,899 bytes in size.

### 1. FIELD

The present invention relates generally to the field of molecular biology. More particularly, it concerns methods and systems for identifying and quantifying nucleic acid targets in biological samples.

### 2. DESCRIPTION OF RELATED ART

Digital assays were developed as a means to accurately quantitate biological analytes in a sample. Generally, performing digital assays requires partitioning the sample so that the average number of copies of an analyte in each partition is less than one. Practically, this means that most partitions will contain either zero or one copy of the analyte of interest, whilst some partitions may contain two, three or more copies of the analyte of interest. The probability of finding zero, one, two or more copies of the analyte in each partition for a given analyte concentration may be described by a Poisson distribution and the concentration of the analyte in the sample may thus be determined by counting the number of partitions containing the analyte relative to the number of partitions that do not contain the analyte.

Detection of nucleic acid analytes in digital PCR (“dPCR”) assays typically makes use of labeled probes or intercalating dyes that emit a fluorescent signal. Analysis of more than one different analyte in a dPCR partition has been achieved through the use of probes labeled with distinguishable fluorophores. However, there are limits to the number of distinguishable fluorophores and detection channels available for use in such systems that typically restrict the number of different analytes that can be detected in a partition to about 3 or 4. This has limited the application of dPCR assays in situations in which a plurality of different analytes are required to be detected in the same partition, such as, for example, in genotyping applications.

In order to achieve higher order multiplexing that permits detection of different analytes in a single detection channel using probes labeled with the same fluorophore, amplitude-based multiplexing has been suggested (Whale et al. Fundamentals of Multiplexing with Digital PCR. Biomolecular Detection and Quantification (2016) 10:15-23). Amplitude-based multiplexing requires assay conditions (such as primer or probe concentrations) to be adjusted to generate signals of a unique, distinguishable predetermined amplitude for each analyte. This concept is based on adjusting amplification efficiency for different targets such that their endpoint fluorescence values are distinguishable on a 1D amplitude plot. When combined with distinguishably labeled probes, measured signals can be represented on 2D plots in which fluorescence amplitude measured in one detection channel is plotted against fluorescence amplitude measured in another detection channel to distinguish different target analytes and thus increase plex level. A drawback with this method is that some targets are deliberately amplified with less than maximal efficiency and optimizing assay conditions to achieve distinguishable amplitudes is time consuming. Furthermore, variances in fluorescence amplitude, particularly at high amplitude levels, can make distinguishing partitions having different analyte occupancy difficult.

Another suggested method of increasing the number of distinguishable target analytes is probe mixing (Whale et al. Fundamentals of Multiplexing with Digital PCR. Biomolecular Detection and Quantification (2016) 10:15-23). In one example, two probes, each specific for one of a first or second analyte, are labeled with distinguishable fluorophores, while a third probe, specific for a third analyte, is labeled with both fluorophores. By plotting the fluorescence amplitude from each detection channel on a 2 dimensional plot, it is possible to distinguish partitions containing the first, second, third, or some combination of the three analytes, from each other. This approach enables the unique identification of three analytes across eight potential occupancy states using two distinguishable fluorophores, however attempts to increase the number of analytes to more than three results in partition subsets having different occupancy states being incompletely separated on the 2-D plot, thus limiting precise analyte quantification.

Using melt analysis in dPCR to detect more than a single analyte in a partition using a single detection channel has been suggested (Tanaka et al Scientific Reports 2019 9:2626). In this study, molecular beacon probes specific for wild type and mutant genotypes were labeled with the same fluorophore and melt analysis was performed to determine the melting temperature (Tm) of probe/analyte duplexes. It was possible to distinguish partitions containing a copy of the wild type analyte of interest from those containing one of two different mutant analytes of interest, based on different calculated Tms of probes binding to their respective target analytes. This approach required measuring signals at many temperatures in order to determine melt profiles and individual Tms for different probes. Furthermore, no analysis was performed on partitions that were suspected to contain more than one of the target analytes, since no definitive Tm could be calculated for these partitions.

Calculation of Tm with sufficient accuracy to enable distinguishing small differences in Tm typically requires high resolution melt (HRM) analysis. While HRM analysis enables genotyping, there are limits in its applications due to the requirement to take a large number of measurements (typically 80-100 over a 40-50 degree temperature span) as the temperature is increased or decreased. This increases the time taken to perform detection and analysis considerably. Furthermore, the requirement to determine Tm to conclusively identify a target analyte limits the number of distinguishable probes that can be detected in a single channel, since duplex melting (or association) occurs over a range of temperatures. Partition signals arising from melting of different target duplexes are typically excluded from analysis unless such signals can be deconvoluted to identify which targets are contributing to the measured signal and in what proportion.

WO2019/144107 describes an alternate method of performing melt analysis in dPCR that does not require determination of the Tm or plotting a melt profile of a target duplex to determine which analytes are present in a sample. In this method, signal is required to be measured only twice to determine if an analyte is present or absent: once at a temperature that is below the Tm of the target duplex and once at a temperature that is above the Tm of the target duplex. Since the Tms of target duplexes are predetermined, a change in signal that occurs across a predetermined temperature interval can be attributed to the presence of a target duplex known to have a Tm falling within the range of the predetermined temperature interval. However, as previously discussed, since duplex melting/association occurs over a range of temperatures, the number of target duplexes that can reliably be detected is limited to the number of non-overlapping melt profiles that can be established within the range of temperatures used for melt analysis.

It would be desirable to find improved methods for performing digital assays that permit detection of multiple targets that are labeled with the same fluorophore within a partition, that reduces the time required for data collection and analysis, and that improves specificity of the reaction to ensure accurate results.

3. Summary

In some embodiments, the present disclosure provides a method for multiplexed detection in a digital assay, the method comprising the steps of (a) partitioning a sample that may contain one or more of a plurality of different target nucleic acids into a plurality of partitions such that if one or more of the plurality of different target nucleic acids is present in the sample some of the partitions contain none of the target nucleic acids and at least some of the partitions contain one or more of the different target nucleic acids; (b) amplifying the plurality of different target nucleic acids, if present, in the plurality of partitions in the presence of a plurality of different signal generating probes, each different signal generating probe being specific for one of the plurality of different target nucleic acids that may be present in the sample and being distinguishable in the presence of its specific target nucleic acid by having a unique melting temperature (Tm) relative to other different signal generating probes; (c) detecting signals from the different signal generating probes in the plurality of partitions at three or more different predetermined temperatures; (d) calculating at least two relational values between signals detected at the three or more different predetermined temperatures for each of the plurality of partitions; and (e) plotting the at least two relational values against each other to determine which, if any, of the plurality of different targets are present in each of the plurality of partitions.

In some aspects, the at least two relational values are calculated from signals that are detected at three or more successive predetermined temperatures. In some aspects, calculating the at least two relational values comprises calculating ratios of signals detected at the three or more predetermined temperatures. Alternatively, calculating the at least two relational values may comprise calculating differences of signals detected at the three or more predetermined temperatures. In some aspects, the plurality of different signal generating probes are labeled with the same signal-generating label. In some aspects amplifying the plurality of different target nucleic acids includes the following steps: (a) hybridizing the plurality of different signal generating probes to their specific target nucleic acids if present; (b) cleaving the hybridized probes to form truncated probes; (c) hybridizing the truncated probes to respective capture sequences; and (d) extending the hybridized truncated probes to form duplexes having predetermined Tms unique to each different probe. In some aspects, each different probe forms a duplex in the presence of its specific target nucleic acid and emits signal of different intensity in the duplex conformation relative to the single stranded conformation. In some aspects, there is a difference of at least three, or at least five, or at least eight degrees centigrade between each of the three more different predetermined temperatures at which signals are detected. In some aspects the method does not include determining the Tm of any of the plurality of different signal-generating probes from the detected signals. In some aspects the method does not include plotting a melt curve for each of the plurality of different signal generating probes from the detected signals.

In some embodiments, the present disclosure provides a method of distinguishing subsets of partitions in a multiplexed digital assay comprising a plurality of different target nucleic acids and a plurality of different probes, each different probe being specific for a different target nucleic acid and having a unique, predetermined Tm in the presence of its target nucleic acid, and wherein the plurality of different probes are labeled with the same reporter, the method comprising the steps of, for each partition: (a) detecting signals from the reporters of the plurality of different probes at three or more different predetermined temperatures; (b) calculating at least two relational values between signals measured at the three or more different predetermined temperatures; and (c) plotting the at least two relational values against each other to distinguish subsets of partitions containing different combinations of the plurality of different target nucleic acids.

In some aspects, the different subsets of partitions are classified to determine which of the plurality of different target nucleic acids are in each partition subset. In some aspects, each different probe forms a duplex in the presence of its specific target nucleic acid and the reporter emits signal of different intensity in the duplex conformation than in single stranded conformation. In some aspects, the partitions are subjected to an amplification reaction prior to the detecting step, wherein the amplification reaction includes the following steps: (a) hybridizing the plurality of different probes to their specific target nucleic acids if present; (b) cleaving the hybridized probes to form truncated probes; (c) hybridizing the truncated probes to respective capture sequences; and (d) extending the hybridized truncated probes to form duplexes having predetermined Tms unique to each different probe. In some aspects, calculating the at least two relational values comprises calculating ratios of signals detected at the three or more different predetermined temperatures. In some aspects, calculating at the least two relational values comprises calculating differences between signals detected at the three or more different predetermined temperatures. In some aspects, the at least two relational values are calculated from signal detected at successive predetermined temperature intervals. In some aspects, there is a difference of at least three degrees C., or at least five degrees C., or at least eight degrees C. between each of the three or more different predetermined temperatures at which signals are detected. In some aspects, the method does not include determining the Tm of any of the plurality of different signal-generating probes from the detected signals. In some aspects, the method does not include plotting a melt curve for each of the plurality of different signal-generating probes from the detected signals.

In some embodiments, the present disclosure provides a method of quantifying a plurality of different target nucleic acids amplified in a digital assay in the presence of a plurality of different probes, each different probe being specific for one of the plurality of different target nucleic acids and distinguishable from other different probes by having a unique Tm, wherein signals from different probes are collected from a plurality of partitions and comprise the same signal-generating reporter, the method comprising the steps of: (a) performing a melt analysis to detect signals from the plurality of different probes in each partition at three or more different predetermined temperatures; (b) calculating at least two relational values for the signals detected at the three or more different predetermined temperatures for each partition; (c) plotting the at least two relational values against each other to identify subsets of partitions containing the same combinations of different target nucleic acids; and (d) quantifying the plurality of different target nucleic acids.

In some aspects, calculating the at least two relational values comprises calculating ratios of signals detected at the three or more different predetermined temperatures. In some aspects calculating at the least two relational values comprises calculating differences between signals detected at the three or more different predetermined temperatures. In some aspects the at least two relational values are calculated from signals detected at successive predetermined temperatures. In some aspects, there is a difference of at least three degrees C., or at least five degrees C., or at least eight degrees C. between each of the three or more different temperatures at which signals are detected. In some aspects, the method does not include determining the Tm of any of the plurality of different probes from the detected signals. In some aspects, the method does not include plotting a melt curve for each of the plurality of different probes from the detected signals. In some aspects each different probe forms a duplex in the presence of its specific target nucleic acid and emits signal of different intensity in the duplex conformation relative to the single stranded conformation.

In some embodiments, the present disclosure provides a method for multiplexed detection in a digital PCR (dPCR) assay, the method comprising the steps of (a) amplifying one or more of a plurality of different target sequences in a sample distributed across a plurality of partitions in the presence of a plurality of different reporter-labeled probes, each different probe being specific for one of the plurality of amplified different target sequences and having a predetermined Tm, wherein at least two different probes labeled with the same reporter are distinguishable in the presence of their respective target sequences based on having different Tms, and at least two different probes labeled with the same reporter are distinguishable in the presence of their respective target sequences based on having different intensity reporter signals, (b) detecting signals from the plurality of different probes in the plurality of partitions at three or more different predetermined temperatures; (c) calculating at least two relational values for signals detected at the three or more different predetermined temperatures for each of the plurality of partitions; and (d) plotting the at least two relational values against each other to determine which, if any, of the plurality of target sequences are present in each of the plurality of partitions.

In some embodiments, the present disclosure provides a method for multiplexed detection in a digital PCR (dPCR) assay, the method comprising the steps of (a) amplifying by a dPCR procedure one or more of a plurality of different target nucleic acids in a sample distributed across a plurality of partitions, wherein the dPCR procedure utilizes a plurality of different signal generating probes, each different signal generating probe being specific for one of the plurality of different target nucleic acids that may be present in the sample and being distinguishable in the presence of its specific target nucleic acid by having a unique melting temperature (Tm) relative to other different signal generating probes in the dPCR procedure; (b) detecting signals from the different probes in the plurality of partitions at three or more different predetermined temperatures; (c) calculating at least two relational values for signals measured at the three or more different predetermined temperatures for each of the plurality of partitions; and (d) plotting the at least two relational values against each other to determine which, if any, of the plurality of target nucleic acids are present in each of the plurality of partitions.

In some aspects, calculating the at least two relational values comprises calculating ratios of signals detected at the three or more different predetermined temperatures. In some aspects, calculating the at least two relational values comprises calculating differences between signals measured at the three or more different predetermined temperatures. In some aspects, the at least two relational values are calculated from signals measured at successive predetermined temperatures. In this embodiment, all different probes may be labeled with the same signal-generating reporter and may be distinguishable either by having unique Tms, and/or by having reporters of different intensities. Alternatively, some probes may be labeled with different signal-generating reporters to increase the number of different targets that can be identified in the assay. In some aspects there is a difference of at least three degrees C., or at least five degrees C., or at least eight degrees C. between each of the three or more different temperatures at which signals are detected. In some aspects, the method does not include determining the Tms of the plurality of different probes from the detected signals or plotting a melt curve for each of the plurality of different probes from the detected signals. In some aspects, each different probe forms a duplex in the presence of its specific target nucleic acid and emits signal of different intensity in the duplex conformation than in single stranded conformation.

In some embodiments, the present disclosure provides a system for detecting the presence or absence of two or more different target nucleic acids in a digital assay, the system comprising: (a) a device comprising a plurality of partitions, wherein the device is configured to receive a sample comprising the two or more different target nucleic acids such that different subsets of partitions have different combinations of the two or more different target nucleic acids; (b) a heat source configured to apply heat to the plurality of partitions to cause the plurality of partitions to be subjected to three or more different predetermined temperatures; (c) an imaging system comprising (i) an illumination system configured to illuminate the plurality of partitions with light of a selected wavelength and (ii) a detection system configured to detect signals from the plurality of partitions; (d) a controller configured to (i) operate the heat source to subject the plurality of partitions to the three or more different predetermined temperatures and (ii) operate the imaging system to illuminate the plurality of partitions with light of a selected wavelength and detect signal from the plurality of partitions at the three or more different predetermined temperatures; and (d) a processor configured to (i) determine at least two relational values from the signals detected at the three or more different predetermined temperatures for each partition of the plurality of partitions; (ii) plot the at least two relational values determined for each partition against each other to identify different subsets of partitions having different combinations of the two or more different target nucleic acids; and (iii) determine the presence or absence of the two or more different target nucleic acids from the different subsets of partitions.

In some aspects, the processor is further configured to operate the heat source to perform a nucleic acid amplification reaction prior to subjecting the plurality of partitions to the three or more predetermined temperatures. In some aspects, the processor is further configured to operate the detector to detect signals from the partitions while the nucleic acid amplification is in progress. In some aspects, the subsets of partitions include subsets comprising one, two or none of the two or more different target nucleic acids. In some aspects, the system further comprises a fluid flow unit configured to direct fluids into the plurality of partitions. In some aspects the detection system is configured to detect fluorescent signals. In some aspects, the plurality of partitions comprises droplets or the plurality of partitions comprises microwells on a solid surface.

In some embodiments, the present disclosure provides a method for multiplexed detection in a digital assay, the method comprising the steps of (a) partitioning a sample that may contain one or more of a plurality of different target nucleic acids into a plurality of partitions such that if one or more of the plurality of different target nucleic acids is present in the sample some of the partitions contain none of the target nucleic acids and at least some of the partitions contain one or more of the different target nucleic acids; (b) amplifying the plurality of different target nucleic acids, if present, in the plurality of partitions in the presence of a plurality of different signal generating probes, each different signal generating probe being specific for one of the plurality of different target nucleic acids that may be present in the sample and being distinguishable in the presence of its specific target nucleic acid by having a unique melting temperature (Tm) relative to other different signal generating probes; (c) detecting signals from the different signal generating probes in the plurality of partitions at three or more different preselected temperatures; (d) calculating at least two relational values between signals detected at the three or more different preselected temperatures for each of the plurality of partitions; and (e) comparing the at least two relational values calculated for each partition to respective sets of predefined relational values to determine the identity of different target nucleic acids in each partition.

In some aspects, the different signal-generating probes are labeled with the same signal-generating reporter. The signal-generating reporter may be a fluorophore. In some aspects, calculating the first and second relational values comprises determining ratios of signals detected at the three of more different preselected temperatures. In some aspects, calculating the first and second relational values comprises determining differences between signals detected at the three or more different preselected temperatures. In some aspects, the at least two relational values are calculated from signals measured at successive preselected temperatures. In some aspects, each of the plurality of different probes forms a duplex after amplification in the presence of its target nucleic acid and the duplex conformation has a signal of different intensity than the single stranded conformation. In some aspects, the method does not include calculating Tms or melt profiles of the plurality of different probes. In some aspects, the method further comprises the step of calculating the number of different partitions comprising each different target nucleic acid and quantifying each of the different target nucleic acids.

In some embodiments, the present disclosure provides a method for multiplexed detection in a digital amplification assay, the method comprising the steps of (a) amplifying one or more of a plurality of different target nucleic acids in a sample distributed across a plurality of partitions, wherein the digital amplification assay utilizes a plurality of different signal generating probes, each different signal generating probe being specific for one of the plurality of different target nucleic acids that may be present in the sample and being distinguishable in the presence of its specific target nucleic acid by having a unique melting temperature (Tm) relative to other different signal generating probes in the digital amplification assay; (b) detecting signals from the different probes in the plurality of partitions at three or more different preselected temperatures; (c) calculating at least two relational values for signals measured at the three or more different preselected temperatures for each of the plurality of partitions; and (d) comparing the at least two relational values calculated for each partition to respective sets of predefined relational values to determine which, if any, of the plurality of target nucleic acids are present in each of the plurality of partitions.

In some aspects, the different signal-generating probes are labeled with the same signal-generating reporter and the signal-generating reporter may be a fluorophore. In some aspects, calculating the first and second relational values comprises determining ratios of signals detected at the three of more different preselected temperatures. In some aspects, calculating the first and second relational values comprises determining differences between signals detected at the three or more different preselected temperatures. In some aspects, the at least two relational values are calculated from signals measured at successive preselected temperatures. In some aspects, each of the plurality of different probes forms a duplex after amplification in the presence of its target nucleic acid and the duplex conformation has a signal of different intensity than the single stranded conformation. In some aspects, the method does not include calculating Tms or melt profiles of the plurality of different probes. In some aspects, the method further comprises the step of calculating the number of different partitions comprising each different target nucleic acid and quantifying each of the different target nucleic acids.

In some embodiments, the present disclosure provides a method of identifying which, if any, of a plurality of different target nucleic acids are amplified in a digital assay in the presence of a plurality of different probes, each different probe being specific for one of the plurality of different target nucleic acids and distinguishable from other different probes by having a unique Tm after amplification in the presence of its target nucleic acid, wherein signals from different probes are collected from each of a plurality of partitions and comprise the same signal-generating reporter, the method comprising the steps of (a) performing a melt analysis to detect signals from the plurality of different probes in each of the plurality of partitions at three or more different preselected temperatures; (b) calculating temperature-dependent multidimensional data from the detected signals in each partition; (c) comparing the calculated multidimensional data to predefined sets of temperature-dependent multidimensional data; and (d) identifying the plurality of different target nucleic acids that were amplified in each partition by identifying the predefined set of temperature-dependent multidimensional data that corresponds to the calculated multidimensional data for each partition.

In some aspects, the multidimensional data is represented by first and second relational values calculated for each partition from the signals detected at three or more different preselected temperatures. In some aspects, calculating the first and second relational values comprises determining ratios of signals detected at the three of more different preselected temperatures. In some aspects, calculating the first and second relational values comprises determining differences between signals detected at the three or more different preselected temperatures. In some aspects, the at least two relational values are calculated from signals measured at successive preselected temperatures. In some aspects of the method, the different target nucleic acids are amplified prior to performing the melt analysis. In some aspects, each of the plurality of different probes forms a duplex after amplification in the presence of its target nucleic acid and the duplex conformation has a signal of different intensity than the single stranded conformation. In some aspects, the method does not include calculating Tms or melt profiles of the plurality of different probes. In some aspects, the method further comprises the step of calculating the number of different partitions comprising each different target nucleic acid and quantifying each of the different target nucleic acids.

In some embodiments, the present disclosure provides a method of identifying combinations of different target nucleic acids amplified in partitions of a multiplexed digital amplification assay comprising a plurality of different target nucleic acids and a plurality of different probes, each different probe being specific for a different target nucleic acid and having a unique, predetermined Tm in the presence of its target nucleic acid, and wherein the plurality of different probes are labeled with the same signal-generating reporter, the method comprising the steps of, for each partition (a) detecting signals from the reporters of the plurality of different probes at three or more different preselected temperatures; (b) calculating temperature-dependent multidimensional data from the detected signals; (c) comparing the calculated multidimensional data for each partition to predefined sets of temperature-dependent multidimensional data; and (d) identifying the combination of different target nucleic acids amplified in each partition when the calculated multidimensional data corresponds to a predefined set of temperature-dependent multidimensional data.

In some aspects, the multidimensional data is represented by first and second relational values calculated for each partition from the signals detected at three or more different preselected temperatures. In some aspects, calculating the first and second relational values comprises determining ratios of signals detected at the three of more different preselected temperatures. In some aspects, calculating the first and second relational values comprises determining differences between signals detected at the three or more different preselected temperatures. In some aspects, the at least two relational values are calculated from signals measured at successive preselected temperatures. In some aspects of the method, the different target nucleic acids are amplified prior to performing the melt analysis. In some aspects, each of the plurality of different probes forms a duplex after amplification in the presence of its target nucleic acid and the duplex conformation has a signal of different intensity than the single stranded conformation. In some aspects, the method does not include calculating Tms or melt profiles of the plurality of different probes. In some aspects, the method further comprises the step of calculating the number of different partitions comprising each different target nucleic acid and quantifying each of the different target nucleic acids.

In some embodiments, the present disclosure provides a system for detecting the presence or absence of two or more different target nucleic acids in a digital assay, the system comprising (a) a device comprising a plurality of partitions, wherein the device is configured to receive a sample comprising the two or more different target nucleic acids such that different subsets of partitions have different combinations of the two or more different target nucleic acids; (b) a heat source configured to apply heat to the plurality of partitions to cause the plurality of partitions to be subjected to three or more different preselected temperatures; (c) an imaging system comprising (i) an illumination system configured to illuminate the plurality of partitions with light of a selected wavelength and (ii) a detection system configured to detect signals from the plurality of partitions; (d) a controller configured to (i) operate the heat source to subject the plurality of partitions to the three or more different preselected temperatures and (ii) operate the imaging system to illuminate the plurality of partitions with light of a selected wavelength and detect signal from the plurality of partitions at the three or more different preselected temperatures; (e) a processor configured to (i) determine at least two relational values from the signals detected at the three or more different preselected temperatures for each partition of the plurality of partitions; (ii) compare the at least two relational values determined for each partition to predefined sets of relational values representative of different combinations of the two or more different target nucleic acids; and (iii) determine the presence or absence of the two or more different target nucleic acids in each partition.

In some aspects, the processor is further configured to operate the heat source to perform a nucleic acid amplification reaction prior to subjecting the plurality of partitions to the three or more preselected temperatures. In some aspects, the processor is further configured to operate the detector to detect signals from the partitions while the nucleic acid amplification is in progress. In some aspects, the system further comprises a fluid flow unit configured to direct fluids into the plurality of partitions. In some aspects, the plurality of partitions comprises droplets. Alternatively, the plurality of partitions may comprise microwells on a solid surface. In some aspects, the subsets of partitions include subsets comprising one, two or none of the two or more different target nucleic acids. In some aspects, the detection system is configured to detect fluorescent signals. Determining the at least two relational values may comprise calculating ratios of signals detected at the three or more preselected temperatures or determining the at least two relational values may comprise determining differences between signals detected at the three or more preselected temperatures. In some aspects, the at least two relational values are calculated from signals measured at successive preselected temperatures.

### 4. BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-1B: (FIG. 1A) Exemplary plot showing the negative derivative of Relative Fluorescence Units (RFU) versus temperature for 3 target duplexes having non-overlapping melt profiles; (FIG. 1B) Exemplary 1D amplitude plot of ratios of RFU measured at four different temperatures, T1, T2, T3 and T4 for 3 partitions where each partition has a different one of the 3 target duplexes of FIG. 1A.

FIGS. 2A-2E: (FIG. 2A) Exemplary plot in which the negative derivative of RFU versus temperature (melt curve) for partitions each containing one of 5 different target duplexes having overlapping melt profiles are overlaid on a single set of axes; (FIG. 2B) Exemplary 1D amplitude plot of ratios of RFU measured at 4 different temperatures, T1, T2, T3 and T4 for 5 partitions where each partition has a different one of the 5 target duplexes of FIG. 2A; (FIG. 2C) Exemplary plot in which the negative derivative of RFU versus temperature (melt curve) for partitions containing all possible combinations of the 5 different target duplexes in FIG. 2A are overlaid on a single set of axes. The melt curves obtained for single occupancy partitions containing one of target duplexes 13, 14, 15 and 16 are shown in solid lines and melt curves obtained for multiple occupancy partitions are shown in dashed lines; (FIG. 2D) Exemplary 1D amplitude plot of ratios of RFU measured at T1, T2, T3 and T4 for all 32 occupancy subsets for the 5 target duplexes shown in FIG. 2A; (FIG. 2E) Exemplary 1D amplitude plot of differences of RFU measured at T1, T2, T3 and T4 for all 32 occupancy subsets for the 5 target duplexes shown in FIG. 2A.

FIGS. 3A-3B: (FIG. 3A) Exemplary 2-dimensional (2D) plots showing calculated differences of RFU measured at 4 different temperatures (T1, T2, T3 and T4) plotted against each other for all possible occupancy subsets of the 5 different target duplexes of FIG. 2A; (FIG. 3B) Exemplary 3-dimensional (3D) plot of the differences determined in FIG. 3A showing all 32 occupancy subsets.

FIGS. 4A-4B: (FIG. 4A) Graphic showing the expected location of subsets of partitions having one of 8 possible occupancies derived from 3 target duplexes (S1, S2 and S3) in plots of differences of RFU measured at 4 different temperatures (T1, T2, T3 and T4), and plotted against each other; (FIG. 4B) Exemplary 3D plot of the differences determined in FIG. 4A showing all 8 occupancy subsets.

FIG. 5: Flow chart depicting the steps of an embodiment of the method.

FIGS. 6A-6E: (FIG. 6A) Exemplary plot in which the negative derivative of RFU versus temperature (melt curve) for partitions each containing one of 6 different target duplexes having one of 3 different Tms are overlaid on a single set of axes. Target duplexes having the same Tm are distinguishable based on their different fluorescence amplitude (18 and 19) which is distinguishable from the fluorescence amplitude of a partition containing both duplexes (20); (FIG. 6B) Exemplary 1D plot of ratios of RFU measured at 4 different temperatures T1, T2, T3 and T4 for all possible 64 partition occupancy subsets from the 6 target duplexes of FIG. 6A; (FIG. 6C) Exemplary 1D plot of differences of RFU measured at 4 different temperatures T1, T2, T3 and T4 for all possible 64 partition occupancy subsets from the 6 target duplexes of FIG. 6A; (FIG. 6D) Exemplary 2D plots showing RFU differences for successive temperature intervals when RFU is measured at 4 different temperatures (T, T2, T3 and T4) plotted against each other for all possible occupancy subsets of the 6 different target duplexes of FIG. 5A; (FIG. 6E) Exemplary 3D plot of the RFU differences determined in FIG. 6D showing all 64 occupancy subsets.

FIGS. 7A-7E: (FIG. 7A) Exemplary plot in which the negative derivative of RFU versus temperature (melt curve) for partitions each containing one of 2 different target duplexes (21, 22) having different Tms or one of 2 different amplified products (23, 24). Amplified product 23 has the same Tm as target duplex 21, while amplified product 24 has the same Tm as target duplex 22; (FIG. 7B) Exemplary 1D plot of ratios of RFU measured at 4 different temperatures T1, T2, T3 and T4 for all possible 16 partition occupancy subsets from the 2 target duplexes and 2 amplification products of FIG. 7A; (FIG. 7C) Exemplary 1D plot of differences of RFU measured at 4 different temperatures T1, T2, T3 and T4 for all possible 16 partition occupancy subsets from the 2 target duplexes and 2 amplification products of FIG. 7A; (FIG. 7D) Exemplary 2D plots showing RFU differences for successive temperature intervals when RFU is measured at 4 different temperatures (T, T2, T3 and T4) plotted against each other for all possible occupancy subsets of the 2 different target duplexes and 2 amplification products of FIG. 7A; (FIG. 7E) Exemplary 3D plot of the differences determined in FIG. 7D showing all 16 occupancy subsets.

FIGS. 8A-8C: (FIG. 8A) Predicted melt derivative curves for three cleavable probes (Target 1, Target 2, Target 3) in the presence of their respective targets. Dashed lines indicate the three temperatures, T1, T2 and T3 at which fluorescence was measured for discrete melt analysis after performing a digital PCR assay; (FIG. 8B) 1D amplitude plots of ratios of RFU measured at T2/T1 and T3/T2 showing a lack of clear separation between the possible 8 occupancy subsets of partitions; (FIG. 8C) 2D plot of ratios calculated for T2/T1 against ratios calculated for T3/T2 showing the separation and identification of 8 different occupancy subsets.

### 5. DETAILED DESCRIPTION

The present disclosure provides methods and systems for performing rapid melt analysis in partitions of digital amplification assays to determine and quantitate the presence or absence of a plurality of different targets within a partition without determining the Tm or plotting a melt profile of individual target duplexes. The methods may be useful when target duplexes have melting temperatures (Tms) that are similar enough to result in overlapping melt profiles across at least some temperatures at which signal is detected. The methods enable multiplexed detection of a plurality of different targets within a partition utilizing a single reporter, thus increasing the number of different targets that may be detected by applying the method across different reporter detection channels. In an embodiment of the method, temperature-dependent reporter signals from two or more target duplexes that may be present in a partition are detected at three or more different predetermined temperatures and used to generate multidimensional data that identifies the target nucleic acids in the partition. The multidimensional data comprises at least two relational values calculated from signals detected at at least two temperature intervals. The first relational value may be compared the second relational value to identify the relationship between the first and second dimensions, which is used to identify the target analytes present in a partition and further, to quantify the different target analytes within the sample.

As used herein “nucleic acid” generally refers to a polymeric form of nucleotides of any length (e.g. at least 2, 3, 4, 5, 6, 10, 50, 100, 200, 500 or 1000 nucleotides), either deoxyribonucleotides or ribonucleotides or a combination thereof, and any modifications thereof. Modifications include, but are not limited to, those that provide other chemical groups that incorporate additional charge, polarizability, hydrogen bonding, electrostatic interaction, and fluxionality to the nucleic acid ligand bases or to the nucleic acid ligand as a whole. Accordingly, the nucleic acids described herein include not only the standard bases adenine (A), cytosine (C), guanine (G), thymine (T), and uracil (U) but also non-standard or non-natural nucleotides, analogs and derivatives thereof. Non-standard or non-natural nucleotides such as isoC or isoG, are described, for example, in U.S. Pat. Nos. 5,432,272, 5,965,364, 6,001,983, 6,037,120, and 6,140,496, all of which are incorporated herein by reference and include bases other than A, G, C, T, or U that can be incorporated into a growing nucleic acid strand by a polymerase and are capable of base-pairing with a complementary non-standard or non-natural nucleotide to form a base pair.

As used herein, the term “sample” generally refers to any material containing or suspected of containing a nucleic acid. A sample may include a bodily tissue or a bodily fluid including but not limited to blood (or a fraction of blood, such as plasma or serum), lymph, mucus, tears, urine, and saliva. A sample may comprise DNA (e.g., genomic DNA), RNA (e.g., mRNA), and/or cDNA, any of which may be amplified to provide an amplified nucleic acid. A sample may comprise material obtained from an environmental locus (e.g., a body of water, soil, and the like) or material obtained from a fomite (i.e., an inanimate object that serves to transfer pathogens from one host to another).

As used herein, “analyte”, “target,” “target sequence” or “target nucleic acid” refers to a nucleic acid sequence of interest. A “target”, “target sequence”, “target nucleic acid” or “target duplex” may also be a surrogate nucleic acid sequence that is formed in the presence of a nucleic acid sequence of interest in a sample and is representative of the nucleic acid sequence of interest. The surrogate nucleic acid sequence may be a probe that forms a duplex having a predetermined Tm in the presence of its target nucleic acid.

An oligonucleotide is a nucleic acid that includes at least two nucleotides. Oligonucleotides used in the methods disclosed herein typically include at least about ten (10) nucleotides and more typically at least about fifteen (15) nucleotides. An oligonucleotide may be designed to function as a “primer.” A “primer” is a short nucleic acid, usually a ssDNA oligonucleotide, which may be hybridized to a target nucleic acid by complementary base-pairing. The primer may then be extended along the target nucleic acid template strand by a polymerase enzyme, such as a DNA polymerase enzyme or an RNA polymerase enzyme. Primer pairs can be used for amplification (and identification) of a target nucleic acid sequence (e.g., by the polymerase chain reaction (PCR)). An oligonucleotide may be designed to function as a “probe.” A “probe” refers to an oligonucleotide or portions thereof, used to detect complementary target nucleic acid sequences. Probes or primers may include a detectable label. Probes may also be extended by a polymerase, using a target nucleic acid or self-complementary regions as a template.

An oligonucleotide that is specific for a target nucleic acid will “hybridize” to the target nucleic acid under suitable conditions. As used herein, “hybridization” or “hybridizing” refers to the process by which an oligonucleotide single strand anneals with a complementary strand through base pairing under defined hybridization conditions. “Specific hybridization” is an indication that two nucleic acid sequences share a high degree of complementarity. Specific hybridization complexes form under permissive annealing conditions. Permissive conditions for annealing of nucleic acid sequences are routinely determinable by one of ordinary skill in the art and may occur, for example, at 65° C. in the presence of about 6×SSC. Annealing temperatures are typically selected to be about 5° C. to 20° C. lower than the thermal melting point (Tₘ) for the specific sequence at a defined ionic strength and pH. The Tₘ is the temperature (under defined ionic strength and pH) at which 50% of the target sequence hybridizes to a perfectly matched probe. Equations for calculating Tₘ, for example, nearest-neighbor parameters, and conditions for nucleic acid hybridization, are known in the art. Melt analysis refers to a process by which hybridization between complementary strands of a nucleic acid duplex is reversed and changes in signal are monitored as the strands dissociate. Starting at or below a temperature at which the duplex is stable, the temperature is increased above the duplex Tm to a temperature at which all duplex molecules become fully dissociated. The hybridization state of the duplex can be monitored through changes in signal such as those from a fluorophore and quencher pair as the duplex dissociates and plotted as a function of relative fluorescence units (RFUs) versus temperature. In a plot of the negative derivative of RFU versus temperature (i.e. a melt curve), the Tm may be calculated from the peak of the curve. Ideally, signals are collected at relatively small temperature increments (for example every 0.5° C.), particularly within close range of the Tm, to generate sufficient data points to calculate an accurate Tm. In some cases, reassociation analysis may be performed rather than melt analysis. In reassociation analysis, the starting temperature is one at which the duplex is dissociated, and signal is measured as the temperature is lowered to a temperature at which the duplex reassociates. While this application uses the term melt analysis to describe the monitoring of the hybridization state of the duplex as the temperature changes, a person skilled in the art understands that reassociation analysis may be substituted for melt analysis in the method of the invention.

As used herein, “amplification” or “amplifying” refers to the production of additional copies of a nucleic acid sequence or a target sequence. Amplification may be carried out using polymerase chain reaction (PCR) or other amplification technologies known in the art, such as isothermal amplification. The term “amplification reaction mixture” refers to an aqueous solution comprising the various reagents used to amplify a target nucleic acid. These may include enzymes (e.g., a thermostable polymerase), aqueous buffers, salts, amplification primers, target nucleic acid, nucleoside triphosphates, and optionally, at least one labeled probe and/or optionally, at least one agent for determining the melting temperature of an amplified target nucleic acid (e.g., a fluorescent intercalating agent that exhibits a change in fluorescence in the presence of double-stranded nucleic acid). In some embodiments, one or more primers or probes in the amplification mixture are labeled with a reporter that emits a detectable signal (e.g., a fluorophore). In some embodiments, the amplification mixture may include at least one nucleotide that is labeled with a quencher (e.g., Dabcyl). In some embodiments the probe may include both a fluorophore and a quencher.

As used herein, “reporters” or “labels” are chemical or biochemical moieties useful for labeling an oligonucleotide or nucleic acid. “Reporters” or “labels” include fluorescent agents, chemiluminescent agents, chromogenic agents, quenching agents, radionuclides, enzymes, substrates, cofactors, scintillation agents, inhibitors, magnetic particles, and other moieties known in the art. “Labels” or “reporters” are capable of generating a measurable signal and may be covalently or noncovalently coupled to an oligonucleotide or nucleotide using methods known in the art.

As used herein, a “fluorescent dye” or a “fluorophore” is a chemical group that can be excited by light to emit fluorescence at a given wavelength or range of wavelengths. Dyes that may be used in the disclosed methods include, but are not limited to, fluorophores such as Alexa Fluor™ dyes, Fluorescein, HEX™ or AquaPhluor® and others known to those skilled in the art.

The oligonucleotides and nucleotides of the disclosed methods may be labeled with a quencher, suitable for quenching fluorescence of a fluorophore. Quenching may include dynamic quenching (e.g., by FRET), static quenching, or both. Suitable quenchers may include Dabcyl and black hole quenchers sold under the trade name “BHQ”.

Digital amplification assays (dAA), as exemplified by digital polymerase chain reaction (dPCR), can be used to directly quantify and clonally amplify nucleic acids, e.g., DNA, cDNA or RNA in a sample. dAA is based on the amplification of a single copy of the target sequence in many separate reactions and involves partitioning the sample such that individual nucleic acid molecules contained in the sample are localized in many separate compartments or partitions prior to amplification. Partitions containing 0 or 1 copy of a given target nucleic acid result in a negative or positive amplification reaction, respectively. This separation allows for a more reliable collection and sensitive measurement of nucleic acid amounts. Accordingly, dAA eliminates the reliance on exponential data to quantify target nucleic acids and provides absolute quantification.

The distribution of target molecules in partitions for dAA follows Poisson statistics such that a statistically significant number of partitions contain zero or one copy of the target sequence, while some partitions will contain two, three, four of more copies of the target sequence. Some applications of dAA require the detection of multiple different target sequences in a partition. The different target sequences may be present on the same target molecule (linked targets) or they may be present on different target molecules (unlinked targets). Accurate quantitation can be performed in either of these scenarios, provided the distribution of each different target in partitions follows Poisson statistics as described above and the detection scheme is capable of independently detecting multiple different targets in the same partition.

Systems for performing dAA include a device suitable for forming a large number of individual reaction compartments. One approach for compartmentalizing reactions is by using droplets, which are isolated volumes of a first fluid that are completely surrounded by a second fluid or by a second fluid and one or more surfaces. In some embodiments, the first and second fluids are two immiscible liquids. In droplet-based biological assays, emulsions may be formed by combining two immiscible phases (e.g., water and oil), often in the presence of one or more surfactants and will typically be a water-in-oil emulsion with the assay reagents (e.g., amplification primers, salts, enzymes, etc.) contained in the aqueous phase. Any suitable non-aqueous fluid may form the non-aqueous continuous phase of the emulsions disclosed herein.

Alternatively, the device may be designed to form compartments or partitions in a static array on a planar surface through, for example, the controlled etching of a silicon, metal or glass or through conventional or microinjection molding techniques. Microfluidic systems that include a fluid flow unit that directs fluids into partitions and subsequently isolates partitions from each other have been described, for example in WO2018094091, incorporated herein by reference. Several different ways of forming static arrays or reaction chambers have been described, for example as in U.S. Pat. No. 9,039,993, PCT/US2003/041356, U.S. Pat. No. 6,391,559, EP2906348, U.S. Pat. No. 9,643,178, and Du. Et al. 2009 “SlipChip.” Lab on a Chip 9 (16):2286, incorporated herein by reference. Optimally, partitions are packed in close proximity to decrease the overall surface area and can be connected by microfluidic channels to improve partition filling. In the latter embodiments, methods such as those described in U.S. Pat. No. 9,163,277 may be used to ensure complete separation of partitions after filling. Further, a gas permeable membrane such as that described in U.S. Ser. No. 10/519,404 may be used.

There are various ways of performing dAA in partitions formed in an emulsion or on a static array. In either case, the target nucleic acid may be diluted to an appropriate concentration, mixed with amplification and detection reagents (primers, dNTPs, polymerase, probes, reporters etc.) and partitioned accordingly into a number of discrete reaction units. Alternatively, partitions may be preformed or preloaded with some or all of the amplification reagents prior to target addition. The partitions are subjected to thermal cycling by a heat source and the target nucleic acids or target duplexes may be detected by monitoring signals from a suitable reporter system (e.g. fluorescence). While PCR is commonly used in dAA, other amplification methods such as isothermal amplification, NASBA or strand displacement amplification may also be used to amplify nucleic acids within partitions. Amplification may be monitored while the amplification reaction is in progress (real-time detection) or after amplification is complete (endpoint detection), or both during amplification and after amplification.

Systems suitable for performing dAA may include an illumination subsystem for delivering light of various wavelengths to partitions, and may include one or more detectors for detecting reporter signals from reporters within partitions of the device. In order to detect signals from a range of fluorophores that emit fluorescence at different wavelengths, the detector may be configured to detect signals at a range of different wavelengths. Detection of signals from dAA is usually accomplished through acquiring images of a plurality of partitions. Depending on the size and number of partitions in the device, multiple images may be required to collect signals from sufficient partitions to achieve accurate results. In order to improve accuracy of image analysis, it may be necessary to normalize detected signals using a partition-specific value to account for differences in illumination or detection in different partitions or regions of an array. Partition-specific values for use in normalization may be obtained at any temperature in which probes are in an unquenched state (i.e. exhibit maximal signal). For example, the labeled cleavable probes described in WO2019/144107 will display maximal fluorescence prior to sample addition and/or amplification, or after the amplification reaction at a temperature at which all probes are single stranded, for example at 95° C. A passive reference dye may also be used for normalization. To facilitate imaging of droplets, the droplets may be dispersed on a surface of the device such that the droplets are disposed substantially in a monolayer. Images may be acquired at a range of temperatures during or after the amplification reaction. Image analysis is performed by a processor that analyzes the intensity of signals collected from selected partitions at selected temperatures.

Current analysis methods for dAA typically utilize endpoint detection to quantify the presence of target nucleic acids. Detection of amplified target nucleic acids in partitions typically relies on the detection of fluorescent signals resulting from intercalating dyes or target specific probes that generate reporter signals in the presence of their target nucleic acids. Classification of partitions as positive or negative for a target sequence typically relies solely on endpoint fluorescence intensity of a partition being above or below an established threshold. Determining thresholds for positivity and negativity can be confounded by the phenomenon known as “rain” i.e. partitions of intermediate intensity between these two categories. The ability to independently detect multiple different targets in a single partition is generally limited by the number of available fluorescence detection channels. Current dAA systems support detection of 3 or 4 different fluorophores, thus limiting the ability to detect more than this number of different targets in a partition. One way to increase the number of different targets that can be detected is to develop methods to distinguish different targets that are labeled with the same reporter fluorophore.

The use of melt/anneal analysis to identify targets based on unique melt temperatures (Tm) as duplex nucleic acids dissociate or reassociate is well established. Melt analysis using target-specific probes or intercalating dyes that show a measurable temperature dependent change in fluorescence as target duplexes dissociate from wholly duplex to wholly single-stranded conformation (or vice-versa in the case of reassociation analysis) can be useful to distinguish target nucleic acids labeled with the same fluorophore since Tm is dependent on the length and GC content of the duplex nucleic acid.

Efforts to apply melt analysis to dAA in an analogous manner as has been applied in quantitative, real-time PCR (qPCR) have identified potential drawbacks. One of the drawbacks is the time required to acquire images used to measure signal to determine melt profiles and calculate Tms in digital assays. Typically, images of partitions in a digital amplification assay are acquired post amplification. To acquire sufficient data points to perform melt analysis, images must be acquired over a large number (e.g. up to 30,000) of partitions at many different temperatures, for example at 35-70 unique temperatures. With typical exposure times on the order of 1s, and an imaging footprint of about 12.5 mm×12.5 mm, generating a melt profile from signals collected at 0.5° C. temperature intervals may require acquiring at least 70 images for a single sample and image acquisition may take more than 10 minutes per detection channel. In systems capable of processing multiple samples on a single device, this may result in samples being exposed to elevated temperatures for long periods of time, which could have deleterious effects on signal. For example, extended periods of time at optimal anneal temperatures might result in non-specific amplification, and extended periods of time at high temperatures might result in irreversible oligonucleotide damage. Finally, the requirement to acquire numerous images risks exposing samples to significant amounts of illumination, which may result in fluorophore photobleaching and consequently, variability in fluorescence that is unrelated to duplex status. Another potential drawback to using melt analysis in dAA, particularly when interrogating partitions for a plurality of different target nucleic acids using a single fluorophore, is the requirement to calculate Tms using a melt curve to accurately identify and quantify a target. Since Tm represents the temperature at which 50% of the target nucleic acid is double stranded and 50% of the target nucleic acid is single stranded, melt data for any single target nucleic acid is captured over a range of temperatures encompassing the Tm of the target duplex. Partitions comprising multiple different targets labeled with the same reporter having Tms that are close together display overlapping melt curves and it may be difficult to distinguish individual Tms from derivative curves for these partitions. Typically, these multiple occupancy partitions are excluded from the analysis, which increases the number of partitions that must be analyzed to accurately determine the number of different targets present in a sample. However, numerous applications of dAA, for example in precision oncology testing for multiple high prevalence mutations, distinguishing donor and recipient cell-free DNA in transplant panels and gene expression signature determination to name a few, would benefit from being able to identify and quantify multiple occupancy partitions. This is especially true when the targets of interest are linked i.e. present on the same nucleic acid molecule. In this scenario, it is quite possible that most partitions will be positive for a plurality of the targets of interest, so being able to quantify these multiple occupancy partitions and to distinguish partitions having different occupancy is essential to the application. Additionally, the ability to correctly identify and quantify multiple occupancy partitions enables inclusion of endogenous controls in digital assays and allows a variety of sample processing, amplification, fluorescence and melt analysis controls to be included at concentrations suitable for partition-by-partition analysis in multiplexed assays.

One way to reduce the time required for detecting targets in partitions of dAA assays using melt analysis, would be to detect reporter signals at fewer temperatures. However, calculating Tm to positively identify a specific target becomes challenging under these conditions, since the larger the temperature interval between measurements, the lower the likelihood of collecting sufficient data points to accurately calculate Tm.

WO2019/144107 teaches a method of detecting a target using melt analysis without determining a Tm or plotting a melt profile for the target duplex. Discrete melt analysis (DMA) can be performed using the labeled cleavable probes described in WO2019/144107, which undergo cleavage upon hybridization to their cognate target sequence, hybridization of a portion of the cleaved probe to a complementary sequence, and extension to form a duplex having a predetermined Tm. Other probes that are suitable for use in DMA include Molecular Beacons and any probes that show a temperature-dependent change in signal between duplex and single stranded conformations in the presence of their specific target sequences. DMA requires measurement of reporter signals at only two temperatures for each target duplex— one that is below the predetermined Tm of the target duplex and at which all target duplex molecules are in the duplex conformation, and one that is above the predetermined Tm of the target duplex and at which all target duplex molecules are in the single stranded conformation. A difference in signal detected at these two measurement temperatures represents a change in conformation (i.e. from wholly duplexed to wholly single stranded) and signifies the presence of the target of interest. If the Tms of different target duplexes labeled with same signaling fluorophore are sufficiently far apart, signals can be measured at predetermined temperatures at which melt profiles of the different duplexes are known to be non-overlapping. Under these conditions, all probes representative of a specific target are either in the duplex conformation at a first predetermined temperature or in the single stranded conformation at a second predetermined temperature. The first predetermined temperature for a duplex having a higher Tm may co-incide with the second predetermined temperature of a duplex with a lower Tm. Accordingly, in a multiplex assay comprising at least 2 different targets and at least 2 different probes, each one specific for one of the targets, signals are measured at three or more different predetermined temperatures. By measuring signal at the first and second predetermined temperatures for each different target duplex, it is possible to calculate a relational value (such as for example a ratio or a difference) between signals measured at the first and second temperatures. Using relational values calculated for signals measured at different temperatures is a convenient way to reduce noise in the system and distinguish probe-specific signals from non-specific signals such as those originating from auto-fluorescence, optical or electronic noise. Relational values are calculated using signals measured at successive temperatures, i.e. for a signal measured at a first temperature, the relational value is calculated from the signal measured at a second temperature that is the nearest (higher or lower) temperature in the set of predetermined temperatures at which signal is measured. When the melt profiles of target duplexes in a reaction are non-overlapping and signals are measured at predefined temperatures at which a particular target duplex is either in the single strand conformation or the duplex conformation, each calculated relational value is representative of the change in reporter signal from target duplexes specific for only one target in the assay. Thus, if the calculated relational value is above a predetermined threshold, it is indicative of the presence of that target. Since signals are measured at a minimum of three different temperatures, at least two relational values can be calculated from the three signal measurements.

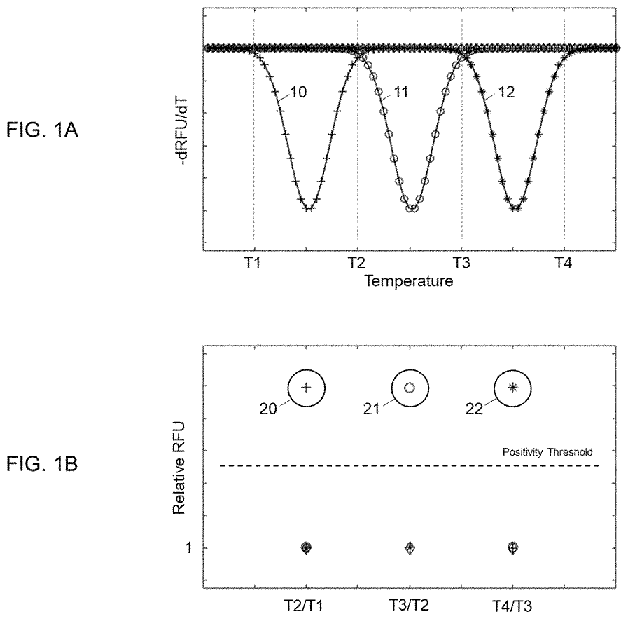

FIG. 1A shows the predicted melt profiles for three different target duplexes, TD1 (10), TD2 (11) and TD3 (12) that have non-overlapping melt profiles. Rather than detecting signal at many temperatures to determine melt profiles and calculate Tms, the DMA method requires that signal be detected at a limited number of temperatures, for example at T1, T2, T3 and T4, temperatures at which all target duplex molecules representative of a particular target are either single stranded or double stranded. Relational values (T2/T1, T3/T2 and T4/T3) for signals measured from each partition at successive temperatures are calculated and represented on a1D amplitude plot (FIG. 1B). TD1 is expected to show a change in fluorescence between T1 and T2, so values>1 at the T2/T1 temperature interval represent partitions positive for this target duplex. Similarly, values>1 are expected at T3/T2 for TD2 and at T4/T3 for TD3. By counting the number of partitions in each cluster (20, 21 and 22) representing each of the different target duplexes TD1, TD2 and TD3 respectively, it is possible to quantify each different target molecule in the sample. In this scenario, each relational value represents reporter signal originating from only a single type of target duplex, since target duplexes are designed not to have overlapping melt profiles.

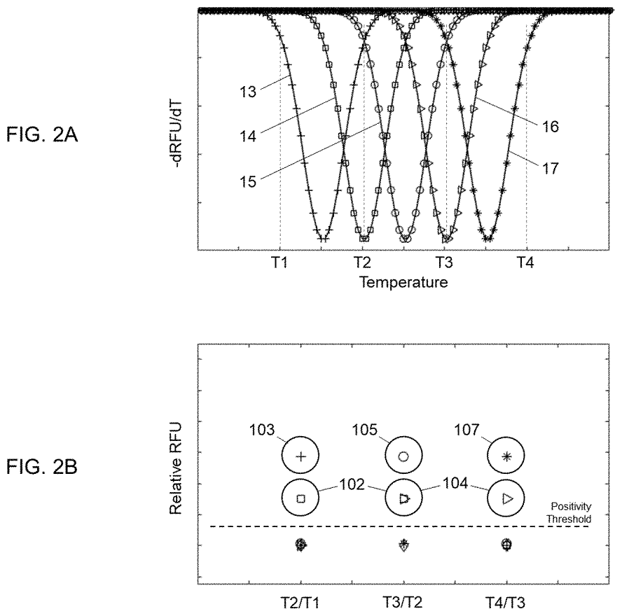

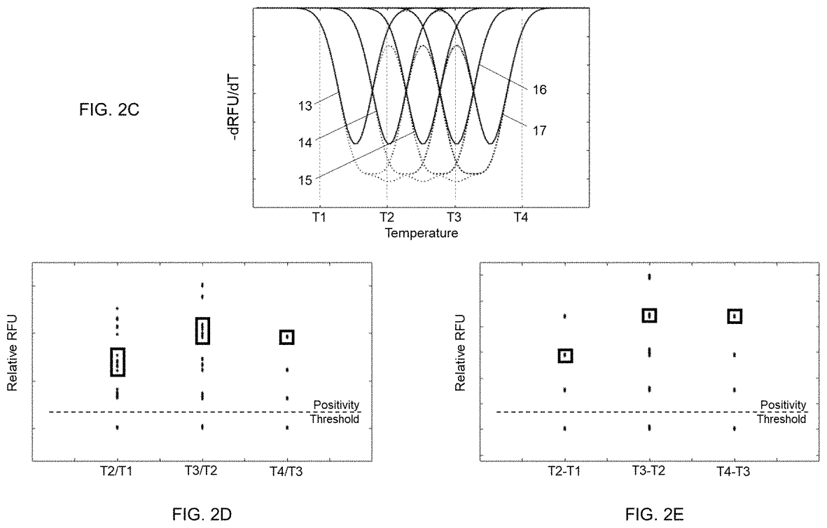

If the number of different probes labeled with the same reporter is increased such that the target duplex melt profiles overlap at any of the temperature intervals at which signals are measured, it becomes difficult to determine what proportion of a measured signal is derived from which target duplex and thus, to determine partition occupancy. FIG. 2A illustrates a scenario in which the predicted melt derivative curve of 5 target duplexes (13, 14, 15, 16 and 17) having predetermined Tms of 60° C., 65° C., 70° C., 75° C. and 85° C. respectively, overlap. This presents a challenge when assigning a signal measured at a specific temperature to the appropriate target duplex, since for most temperatures within the range of Tms at which signal is measured, signal is derived from more than one target duplex. Using the DMA method, signals can be measured at 10° C. intervals over a temperature range of 60° C. to 90° C. (T1, T2, T3 and T4) and relational values (in this case, ratios T2/T1, T3/T2 and T4/T3) of signals measured at successive temperatures are calculated. As shown in the simplified 1D amplitude plot (FIG. 2B) representing signals collected from only 5 partitions that each contain a different target duplex and 1 partition negative for all targets, calculated ratios for some partitions show an expected maximum value relative to the positivity threshold. These represent duplexes that change conformation from wholly duplex to wholly single strand between the two temperatures used to calculate the relational value e.g. Target 13 at T2/T1 (103), Target 15 at T3/T2 (105) and Target 17 at T4/T3 (107). Ratios calculated for other partitions show an intermediate level of positivity at more than one set of temperature intervals e.g. Target 14 at T2/T1 and T3/T2 (102) and Target 16 at T3/T2 and T4/T3 (104). Since target duplexes 14 and 16 are expected to be partially in duplex conformation and partially in single stranded conformation at temperatures T2 and T3, respectively, they contribute partial signal to any ratios calculated using signals measured at T2 and T3 respectively. This is illustrated by the reduction in relational values calculated for Target 14 and Target 16 detected at both T2/T1 and T3/T2, and T3/T2 and T4/T3 respectively in FIG. 2B. In this example, T2 and T3 coincide with the Tms (at which 50% of the target duplex is in duplex conformation and 50% of the target duplex is in single stranded conformation) of target duplexes 14 and 16 respectively, however this may not always be the case. Application of DMA and the use of 1D amplitude plots to associate signals with specific target duplexes (and by inference detect corresponding analytes) may be practical when partitions are not likely to include different target duplexes having overlapping melt profiles. However, in multiplex dPCR assays in which partitions are likely to include more than one different target (either linked or unlinked) and different target duplex melt profiles overlap, it becomes difficult to accurately distinguish clusters of partition subsets containing different combinations of the different target duplexes. As can be seen in FIG. 2B, it may also be difficult to distinguish low amplitude signals that result from target duplex melting occurring over 2 temperature intervals, from rain.

In the scenario in which 5 different target duplexes are used to detect 5 different analytes there are 32 (2⁵) possible different subsets of partition occupancy. FIG. 2C illustrates all possible predicted melt derivative curves that may arise in this scenario. In addition to the overlapping predicted melt derivative curves for targets 13, 14, 15, 16 and 17 obtained from single occupancy partitions as shown in FIG. 2A, many other overlapping melt derivative curves are possible (shown by dashed lines), illustrating the challenge associated with trying to identify and quantify targets in multiple occupancy partitions using traditional melt curve analysis. Applying the DMA principle to a set of partitions representing all possible 32 occupancy subsets by measuring signals at a limited set of predetermined temperatures (T1, T2, T3 and T4), one can calculate relative values (ratios or differences) of signals measured at successive temperatures for each partition. FIG. 2D is a 1D amplitude plot of ratios of fluorescence intensity (T2/T1, T3/T2 and T4/T3) calculated for signals measured at the four successive temperatures while FIG. 2E is a 1D amplitude plot of differences in fluorescence intensity calculated for signals measured at the four successive temperatures. Despite the fact that FIG. 2D and FIG. 2E represent a simplified (i.e. only 1 partition per occupancy subset) and highly idealized (in terms of relative duplex Tms) dataset, it is evident that distinguishing subsets of partitions containing different combinations of the different targets from 1D plots becomes challenging. This is especially true for partition subsets containing target duplexes that are not wholly in the duplex or single stranded conformation at any temperature at which signal is measured, since these will contribute signal at multiple sets of ratios. In FIGS. 2D and 2E, relative intensity values associated with partitions containing all four targets 13, 15, 16 and 17 fall within the area enclosed by a rectangle. However, distinguishing this partition occupancy subset from other subsets having similar relative intensity values becomes challenging as relative intensity values for different occupancy subsets may overlap. Adding to the complexity is the fact that there is some variability in the calculated ratio or difference for partitions within a given subset containing a given combination of targets. In reality, when signals from thousands of partitions are plotted, the separation between subsets in the 1D amplitude plot is reduced, thus increasing the risk of misclassifying a partition into the incorrect occupancy subset. Furthermore, low amplitude values may be confused with rain and vice versa. While this variability can be somewhat reduced (as shown in FIG. 2E) by using the difference in signals rather than ratios (particularly if signals are normalized by a partition-specific value to account for non-uniformity of measureable signals across the partition array), reliable separation of partition subsets is still challenging in practice when a large number of data points are required to obtain accurate results.

The method disclosed herein is designed to distinguish temperature dependent signals arising from different target duplexes (or combinations of target duplexes) in a partition of a dAA when all target duplexes are labeled with the same reporter, without having to calculate a Tm or melt profile for the different target duplexes, Each different target duplex representative of a different target nucleic acid, has a unique Tm and melt profile relative to other different target duplexes present in the partition, but some melt profiles may overlap at temperatures at which signals are measured, such that multiple duplexes contribute to a detected signal at a selected temperature. Notably, because the method does not require the calculation of Tms for distinct duplexes (and by extension the acquisition of signals at a sufficient number of different temperatures to enable generation of melt derivative plots), the time required for signal acquisition and data analysis in dAA is significantly reduced when using the method of the invention.

While different target duplexes labeled with the same reporter may have overlapping melt profiles, provided each different target duplex has a unique Tm (and consequently, a unique melt profile), each different target duplex will contribute different proportions of signal at a selected temperature. In order to correctly classify partitions into the appropriate occupancy subset, it is necessary to unambiguously identify corresponding relational values representative of a particular occupancy subset in more than one temperature interval. Similarly, when the preselected temperatures at which signal is detected for a first temperature interval do not correspond to temperatures at which a particular target duplex population is wholly single stranded or wholly in the duplex conformation, only a portion of the maximum expected change in signal intensity will be detected at the first temperature interval. The change in signal intensity associated with dissociation of the remaining target duplex for the population may be detected at a second temperature interval. For each of the scenarios described above, a particular target duplex contributes signal in at least two temperature-dependent dimensions, where each dimension is a relational value calculated from signal detected at two successive temperatures. By utilizing probe populations specific for different targets that form target duplex populations with unique Tms in the presence of their cognate target nucleic acids, it is possible to distinguish the identity of target duplexes contributing to a particular set of signals detected in a partition by utilizing the multidimensional nature of the detected signals. For each different combination of target duplexes in a partition, a first relational value determined from signal detected at a first temperature interval will be correlated to a second relational value determined from signal detected at a second temperature interval. Accordingly, the multidimensional nature of the first and second relational values can be correlated to particular combinations of target nucleic acids on a per partition basis, by using a function that defines the relationship between the two relational values, or that defines a multidimensional shape and position representative of each combination of target duplexes.

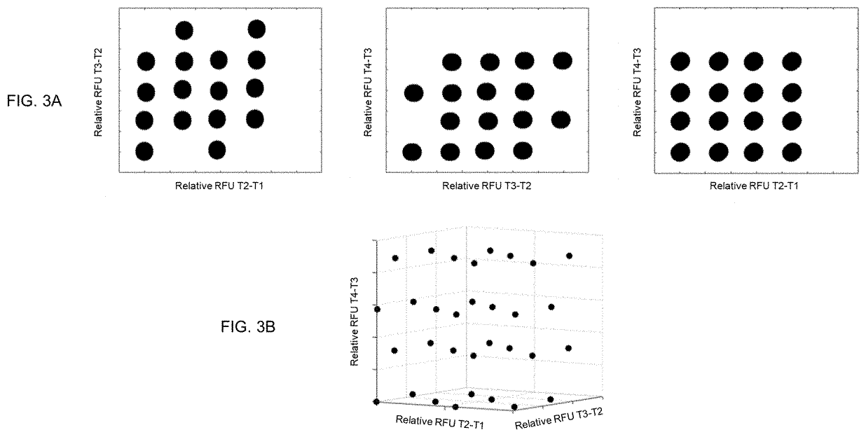

In one embodiment of the method, plotting the relational values calculated for signals measured at three or more successive predetermined temperatures against each other provides an improved means of grouping subsets of partitions of different occupancy into distinguishable clusters. By understanding the contribution of individual target duplexes to each relational value, one 'can predict the location of a partition containing any combination of target duplexes on a 2 or 3-dimensional plot of relational values. Accordingly, once subsets are grouped into distinguishable clusters of assigned occupancy on such a plot, it becomes possible to accurately quantify each of the different target nucleic acids in the sample. The calculated relational values may be plotted in 2-dimensional plots, or in some instances, in 3-dimensional plots. In the examples illustrated in FIG. 2 in which there are 5 different target duplexes each with a unique Tm, signals are measured at temperatures T1, T2, T3 and T4, and RFU differences are calculated for intervals T2-T1, T3-T2 and T4-T3. Three two dimensional plots of RFU differences calculated for each partition at these intervals represents all 32 possible occupancy states (FIG. 3A) for partitions and permits the identification of clusters of partitions having the same occupancy, and subsequent quantitation of target analytes. Alternatively, the calculated differences can be plotted on a three dimensional plot to identify all possible 32 occupancy states (FIG. 3B). In general, the method requires measurement of signals at three or more different temperatures in order to calculate at least two relational values that can be plotted against each other.

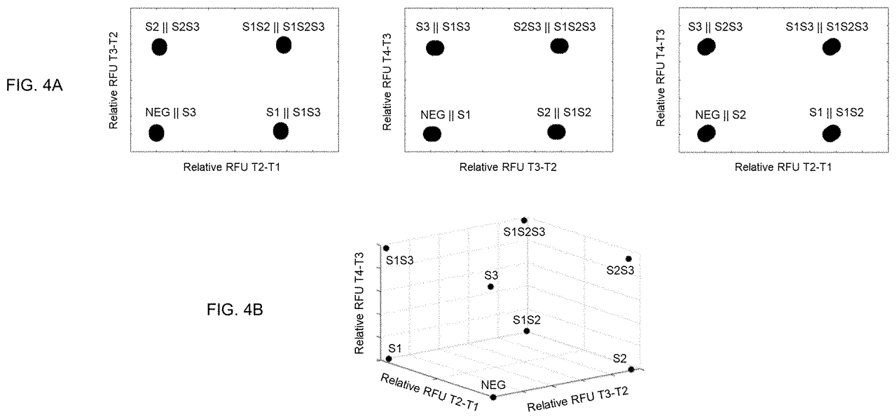

Calculation of the number of different target molecules in the sample may be performed using Boolean logic. Consider a dAA assay in which three unique target sequences may be present in any combination (2³ possible combinations) in individual partitions. The assay includes three different target specific probes, each capable of forming a different target duplex (S1, S2 or S3) with a unique Tm in the presence of its target nucleic acid, and all labeled with the same reporter. After partitioning and amplification in the presence of the different probes, melt analysis is performed and signals are measured at 4 temperatures (T1, T2, T3 and T4). RFU differences for signals measured at successive temperatures are calculated for each partition and represented on three 2-dimensional plots. Eight different occupancy states are possible in this scenario, namely Negative, S1, S2, S3, S152, S153, S2S3 and S1S2S3 and are represented in the clusters of partition subsets in the three 2-dimensional plots shown in FIG. 4A.

By applying Boolean Logic, an example of which is shown below, it is possible to quantify each of three targets.

### NEG=A1 & A2 & A3

### S1=A2 ! NEG

### S2=A3 ! NEG

### S3=A1 ! NEG

### S1 S2=D2 ! S2 or S1 S2=D3 ! S1

### S1 S3=D1 !S1 or S1 S3=B2 ! S3

### S2S3=B1 ! S2 or S2S3=B3 ! S3

### S1 S2S3=C1 ! S1 S2 or

### S1 S2S3=C2 ! S2S3 or

### S1 S2S3=C3 ! S1 S3

Alternatively, a 3D plot (FIG. 4B) may be used to cluster the 8 subsets of partitions of different occupancy and quantify the number of target molecules in the sample. Depending on the number of temperatures at which signal is measured, multiple 3D plots may be necessary to ensure relational values for each temperature interval are plotted against relational values for every other temperature interval.

In another embodiment, generating a 2D or 3D plot of the relational values is not required. Instead, a computational control flow algorithm based on comparison and/or Boolean operators may be used to determine the identity of target molecules in a partition. For example, minimum and maximum thresholds for each of the at least two relational values can be predetermined for each different target duplex. In this way, each relational value for each different target duplex is associated with a predefined range of values. If the relational value calculated from signal intensities measured at the two temperatures defining a temperature interval falls within the predefined range set for a particular target duplex, the relational value can be defined as true. In this example, application of a Boolean logic operator that defines a specific target to be true/present when specific criteria are met (i.e. the at least two calculated relational values fall within the predefined ranges for a particular target duplex) can be used to identify the presence of a particular target.

As noted previously, when using the cleavable probes described in WO2019/144107, a maximum change in signal intensity occurs when signals detected at a first temperature at which all target duplexes of a population are in duplex conformation are compared to signals measured at a second temperature at which all target duplexes are in single stranded conformation. However, in some cases, the first and second temperatures defining the temperature interval do not correspond to conditions in which target duplexes of the population are either all in single stranded conformation or all in duplex conformation. Under these conditions, the first relational value calculated for a first temperature interval represents only a portion of the expected maximum change in signal intensity that occurs when all target duplexes of the population melts. The change in signal intensity associated with dissociation of the remaining target duplexes of the population may be detected in a second relational value calculated for a second temperature interval. Thus, the maximum change in signal intensity is detected across two relational values. By comparing first and second relational values calculated from signal intensities detected at first and second temperature intervals to a predefined range of corresponding values for the target duplex and determining if the values fall within the predefined ranges, it is possible to determine the identity of the target duplex associated with the change in signal in each partition.

When multiple different targets are present in a partition of a dAA and target duplex melt analysis is used to identify which targets are present on a per partition basis using the method of the invention, relational values calculated at one or more temperature intervals may represent changes in signal intensity from more than one different target duplex. This occurs when two or more different target duplexes show a change in signal intensity at the same temperature interval. In these instances, the relational value represents the sum of relational values calculated for the two or more different target duplexes. In this embodiment of the method for identifying targets present in a partition, at least two calculated relational values can be compared to respective predefined ranges representing the expected cumulative relational values for each of at least two temperature intervals to determine the identity of target duplexes present in a partition and further, to quantify the targets in the sample. Thus, more than one set of predefined ranges may be established for each relational value wherein different ranges represent different combinations of targets that may be present in a partition. A computational algorithm may be used to compare the first calculated relational value with all possible predefined relational values for the first temperature interval to identify possible partition occupancy subsets. The algorithm then compares the second calculated relational value with all possible predefined second relational values correlated to the predefined range of first relational values and identifies the correct occupancy subset. The ranges of values defining the correlation between the predefined first and second relational values may comprise data frames, data tables, strings, arrays, matrices, sets, graphs or lookup tables. A comparison of at least two relational values calculated for at least two sets of temperature intervals with the predefined ranges for different combinations of targets for the respective temperature intervals, permits the identification of which combination of targets is present in a partition.

In the aforementioned instances where the maximum change in signal intensity as a probe dissociates occurs over two temperature intervals and is reflected in two relational values, or where a single relational value represents changes in signal intensity originating from more than one different probe, the resulting data can be considered to be multidimensional. In each case, signals originating from two or more different target duplexes labeled with the same reporter may be detected in the same temperature interval and must be deconvoluted to determine the extent to which each target duplex contributes to detected signal. While signals from different target duplexes may be indistinguishable in a first dimension (both contribute signal to a first relational value), they can be designed to ensure that their signals are distinguishable in a second dimension (for example, only one contributes signal to a second relational value). Alternatively, if multiple target duplexes each contribute a predefined and different proportion of the cumulative relational value in each dimension/relational value, the multidimensional nature of the data can be used to determine the identity of the target duplexes. Accordingly, for a multiplex dAA in which different target duplexes are labeled with the same reporter, but each different target duplex has a unique melt profile, sets of multidimensional data representing various combinations of targets in a partition can be predefined. The predefined data sets are representative of the expected changes in signal detected at various temperature intervals when a partition contains one of various possible combinations of targets. After amplifying all possible targets in partitions of the dAA, melt analysis is performed and signals are detected at at least three different temperatures. At least two relational values are calculated from the signals detected at the at least three different temperatures and the calculated relational values are compared to the predefined multidimensional data sets. By identifying the predefined data set to which the calculated first and second relational values most closely correspond, the identity of the target(s) present in the partition can be determined.

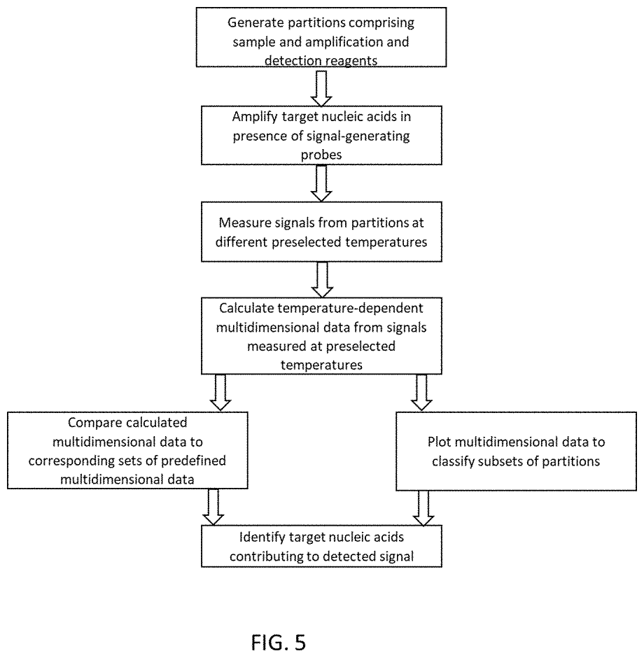

The flowchart shown in FIG. 5 illustrates the steps of the method that can be performed to enable quantitation of multiple target nucleic acids within a sample in a dAA assay using a single reporter. In the first step, partitions comprising the sample and amplification and detection reagents are generated such that the targets in the sample follow a Poisson distribution across the partitions. Following an amplification step, temperature-dependent signals are detected on a per partition basis at different preselected temperatures and multidimensional data calculated. The multidimensional data, represented by at least two calculated relational values, may be plotted in a set of 2D graphs or a 3D graph to separate subsets of partitions comprising the same combination of target nucleic acids into clusters that are distinguishable from other subsets of partitions comprising different combinations target nucleic acids. Alternatively, the multidimensional data, as represented by at least two calculated relational values, may be compared to predefined ranges of values for each respective relational value to identify the combinations of targets in a partition.

Multiplexed melt analysis may also be used in combination with amplitude-based multiplexing in digital amplification assays to further increase the number of different targets that can be interrogated using a single reporter. Amplitude-based multiplexing relies on distinguishing probes that are specific for different targets but labeled with the same reporter by virtue of their distinguishable fluorescence amplitude. Different fluorescence amplitude signals may be achieved through the use of different concentrations of probes or primers, or different intensities of the reporter(s) attached to probes. Amplitude-based multiplexing can be combined with melt analysis based multiplexing using DMA as described herein to further increase single channel multiplex capability in dAA applications. When used in combination with a dAA system capable of detection in multiple channels, this strategy can increase the ability to interrogate a plurality of targets substantially.

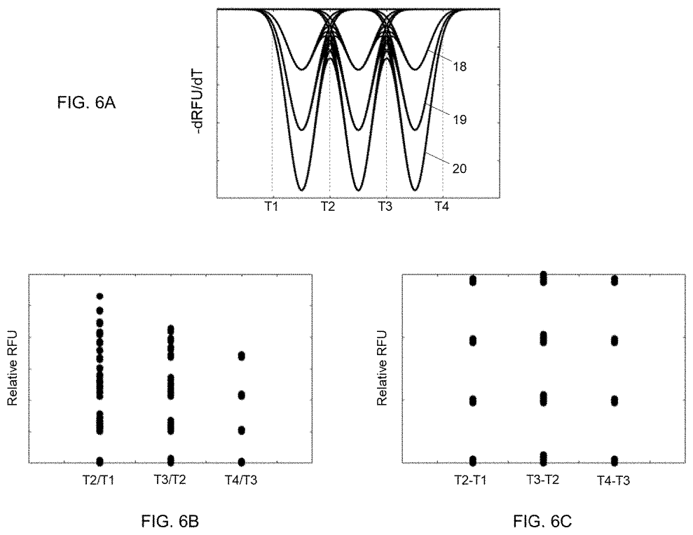

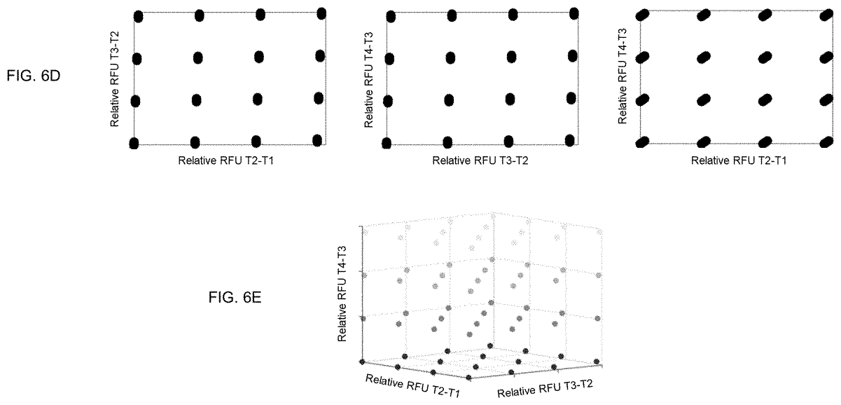

In a dPCR assay in which 6 different targets are interrogated with 6 different probes, each specific for its cognate target sequence, 64 (2⁶) different occupancy subsets are possible in the partitions. Probes are designed to form target duplexes having one of three different Tms, such that two target duplexes have Tₘ1 (65° C.), two target duplexes have Tₘ2 (75° C.) and two target duplexes have TO (85° C.) (FIG. 6A). For different target duplexes sharing the same Tₘ, if one is present at twice the concentration of the other it will have a signal that is twice the maximum amplitude of the other probe. This is represented in the predicted derivative melt curve shown in FIG. 6A by the lower amplitude of the lower concentration target duplex (18) relative to the amplitude of the higher concentration target duplex (19) having the same Tm. A partition containing both target duplexes would show the sum of these two amplitudes (20) due the combined signals from the two target duplexes. In this particular example, melt analysis curves for duplexes that do not share the same Tm do not overlap, however in other instances duplexes having different Tms may show overlapping melt curves. Using a DMA approach, signals are measured at 4 different temperatures, T1, T2, T3 and T4, across the melting temperature range of all target duplexes. Relative values of fluorescence signal measured at successive temperatures are calculated. On a 1D plot representing calculated relative values (ratios or differences) at each successive temperature interval, it becomes almost impossible to distinguish the 64 different occupancy states (FIG. 6B and FIG. 6C). However, when the calculated differences (or ratios) are plotted in 2 or 3 dimensions as shown in FIG. 6D and FIG. 6E, clusters representing all 64 subsets of partitions having the same target occupancy can be easily distinguished and quantified.

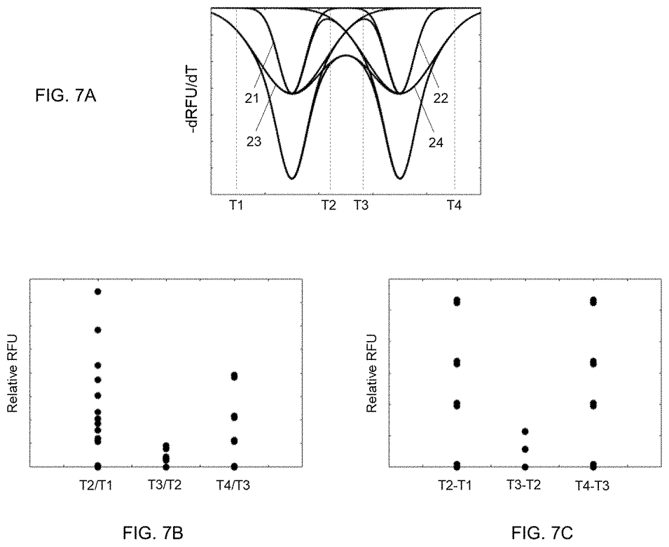

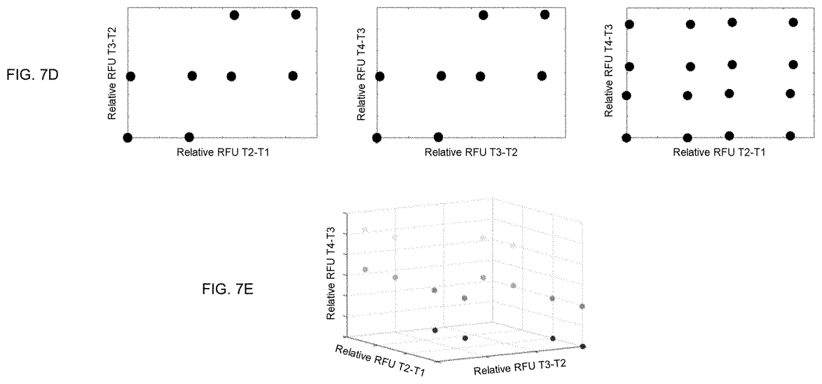

In another variation, DMA may be used in dAA in which some targets are detected using target-specific probes while other amplified targets are detected using intercalating dyes, where detection occurs in a single channel. In the predicted melt curves shown in FIG. 7A, 4 different targets may be present in any combination, so there are a possible 16 different partition occupancy subsets. The first and second targets are detected using first and second target-specific probes labeled with the same reporter but distinguishable from each other by each forming a duplex having a unique Tm (21, 22) in the presence of its cognate target. The third and fourth targets are detected using an intercalating dye that binds to the third and fourth amplification products (23, 24) resulting from the dAA reaction. The third amplification product (23) has a Tm very similar to the Tm of the target duplex of the first detection probe (21), while the fourth amplification product (24) has a Tm very similar to the Tm of the target duplex of the second detection probe (22). Despite each having a similar Tm to a target duplex in the reaction mixture, the third and fourth amplification products have distinguishable predicted melt profiles from the first and second target duplexes, respectively. As can be seen in FIG. 7A, the target duplexes display narrow melt profiles while the amplification products display wide melt profiles. Using DMA, signals can be measured at four different temperatures (T1, T2, T3 and T4) and relational values between measured signals are calculated. FIG. 7B shows a 1D amplitude plot of calculated ratios and FIG. 7C shows a 1D amplitude plot of calculated differences in signals measured at successive temperatures. As the number of partitions increases, the variability in measured signals from partitions having the same occupancy makes it increasingly difficult to distinguish subsets of partitions having different occupancy from each other. However, when the differences (or ratios) are plotted against each other, the 16 different occupancy states can be clearly distinguished on the three different 2D plots (FIG. 7D) or on the 3D plot (FIG. 7E).

A system for detecting the presence or absence of multiple targets in a digital assay includes a device comprising a plurality of partitions, a heat source, an imaging system that includes an illumination subsystem for illuminating the partitions with light of a selected wavelength and a detection system for detecting signals such as fluorescent signals from signal-generating probes present in the partitions. The illumination subsystem may be configured to illuminate the partitions with light of a variety of wavelengths to facilitate the detection of targets labeled with different signal-generating labels. The partitions may be in the form of droplets, or may be microwells on a planar surface. The sample comprising two or more target nucleic acids is distributed across the partitions such that the two or more target nucleic acids are distributed in accordance with Poisson statistics. The system may include a fluid flow control unit for directing the sample into the partitions. The sample may be premixed with amplification and detection reagents prior to distribution into the partitions or the amplification and detection reagents may be distributed into the partitions prior to addition of the sample. The heat source may be operable to heat the contents of the partitions to perform a nucleic acid amplification reaction prior to operation of the imaging system. The imaging system may also be operable to collect images during a nucleic acid amplification reaction.

The operation of the system is controlled by a controller that operates the heat source to subject the partitions to three or more predetermined temperatures and operates the imaging system to collect images representing the intensity of signal emitted from the probes at the three or more predetermined temperatures. The system further includes a processor that determines at least two relational values from the detected signal intensities at the three or more predetermined temperatures and plots the relational values against each other to classify subsets of partitions comprising the same target nucleic acids. The processor may also calculate the copy number of each different target nucleic acid in the sample.

### Example

In this experiment 3 different synthetic (gblock) target nucleic acids (shown in Table 1) representing regions of the ESR-1 gene were amplified, detected and quantified in a digital amplification assay. Three cleavable probes, each one specific for a different one of the three target nucleic acids, and all labeled at their 5′ end with AlphaPhluor® 525 (AP525) reporter were synthesized (Table 2). In addition to the AP525 reporter coupled to a 5′ terminal iso-C nucleotide, the probes contain a synthetic sequence, a target-specific region that includes a ribonucleotide, and a 3′ blocking group. The synthetic sequence includes a loop forming region and a region that is partially self-complementary such that it can form a partial hairpin. In the presence of their specific target nucleic acids, the probes hybridize to their complementary target sequence and in the presence of RNAse HII, the duplex is cleaved at the RNA:DNA hybrid to yield a free 3′-OH end. The cleaved probe is released from the target nucleic acid and forms a self-complementary partial hairpin. Using the synthetic sequence as a template, the free 3′ end is extended by polymerase, incorporating a quencher labeled iso-G nucleotide opposite the reporter labeled iso-C nucleotide. This extended probe forms a target duplex labeled with a reporter-quencher pair and a predetermined Tm that can be used in melt analysis to identify the presence of a specific target nucleic acid. In this particular labeling scheme, target duplexes are quenched in double stranded conformation and exhibit maximum signal when single stranded. Amplification primers (shown in Table 3) are included to amplify the synthetic target nucleic acids.

TABLE 1
Synthetic target (gblock) nucleic acids

| gblock | |
|---|---|
| Name | Sequence |
| TW664 | TTCTGTGTCTTCCCACCTACAGTAACAAAGGCATGGAGCATCTGTACAGCATGAAGT |
| | GCAAGAACGTGGTGCCCCTCTGTGACCTGCTGCTGGAGATGCTGGACGCCCACCGCC |
| | TACATGCGCCCACTAGCCGTGGAGGGGCATCCGTGGAGGAGACGGACCAAAGC |
| | (SEQ ID NO: 1) |
| TW669 | TTCTGTGTCTTCCCACCTACAGTAACAAAGGCATGGAGCATCTGTACAGCATGAAGT |
| | GCAAGAACGTGGTGCACCTCTATGACCTGCTGCTGGAGATGCTGGACGCCCACCGCC |
| | TACATGCGCCCACTAGCCGTGGAGGGGCATCCGTGGAGGAGACGGACCAAAGC |
| | (SEQ ID NO: 2) |
| ACC1 | CTGGTCTTGATTTTTCGTATATTTCAGGAGATAAGTTTTAAAAGTAAGCCGAATGTC |
| | TGGGGATATTTCTCAGTTAAGGTAAGCTGTTCATAGCTCTGTTGCAGTGTCATGTTG |
| | TTTTGAGTTCTGCAGAAAATTGGTCTGCAAGATTTTTTATTTGAATTAGTATTCTC |
| | (SEQ ID NO: 3) |

TABLE 2
Cleavable hairpin probe Sequences

| | | Target |
|---|---|---|
| | | Duplex |
| Probe | Sequence | Tm (° C.) |
| JT435-525-L1 | /AP525//iMe- | 65 |
| | isodC/AAATAAATATATAATAATAACTGACACCAAAA/iSpc3/ | |
| | AAAACCTGTCAGTTArAGGTAAGCTGTTCATAG/3SpC3/ | |
| | (SEQ ID NO: 4) | |
| JT0410-525-M | /AP525//iMe- | 75 |
| | isodC/TCCTCTTCTTCTTCTTTCAGAGGTACCAAAA/iSpC3/ | |
| | AAAAACCTACCTCTGrUGACCTGCTG/3SpC3/ (SEQ ID | |
| | NO: 5) | |
| JB0043-525- | /AP525//iMe- | 85 |
| H3 | isodC/CCCTCCTCCTCCTTCCTCCCTGCACACCAAA/iSpC3/ | |
| | AAACCTGTGCArCCTCTATGACCT/3-MGB/ (SEQ ID NO: | |
| | 6) | |

TABLE 3
Primer sequences used to amplify the targets within the sample mixture

| Primer | Sequence | Reaction concentration |
|---|---|---|
| JT0401 | TGG GCG TCC AGC ATC TC (SEQ ID NO: | 800 nM |
| | 7) | |
| LH1375 | CAA AGG CAT GGA GCA TCT GTA (SEQ | 200 nM |
| | ID NO: 8) | |
| JT0434 | AAC AAC ATG ACA CTG CAA CAG A (SEQ | 800 nM |
| | ID NO: 9) | |
| JT0433 | AAG CCG AAT GTC TGG GGA TAT TTC | 200 nM |
| | (SEQ ID NO: 10) | |

The amplification mixture contained 10 mM Tris, 20 mM BTP, 300 ug/mL BSA, 0.09 mM DTT, 2.5 mM MgCl2, 50 mM KCl, 0.1 mM dNTPs, 1 uM Dabcyl-diGTP, 2×Titanium Taq Polymerase (Takara Biosciences), 44.8 mU/ul RNAseH2 (Takara Biosciences), 200 nM each forward oligonucleotide primer (JT401 and JT434) and 800 nM each reverse oligonucleotide primer (LH1375 and JT435), and 200 nM of each cleavable hairpin probe labeled with AP525 fluorophore. All three targets were present in the reaction at an input of 5,600 copies. Nine microliters of the reaction mixture was transferred to each well of a Combinati MAP16 consumable and covered with 15 microliters of Isolation Buffer (Combinati). Gaskets were appended to each well and the consumable was transferred to the Combinati IQI dPCR system. The consumable was pressurized at 75 psi for 25 minutes and held at 50 psi to load and digitize the sample across approximately 20,000 partitions. The concentration of the 3 targets in the reaction was specifically determined to allow for the possibility of any of the 8 possible occupancy subpopulations to be present in each well after loading. Amplification was performed by subjecting the consumable to 95° C. for 5 minutes, followed by 35 cycles of 5 seconds at 92° C., 20 seconds at 58° C., followed by 30 cycles of 5 seconds at 75° C. and 20 seconds at 56° C., all while holding the pressure at 50 psi.

After thermal cycling, Combinati analysis software was used to locate and identify partitions within each unit and to acquire images of partitions at 60° C. (T1), 76° C. (T2) and 92° C. (T3) with optical filters set to preferentially obtain fluorescence values for the AP525 fluorophore.

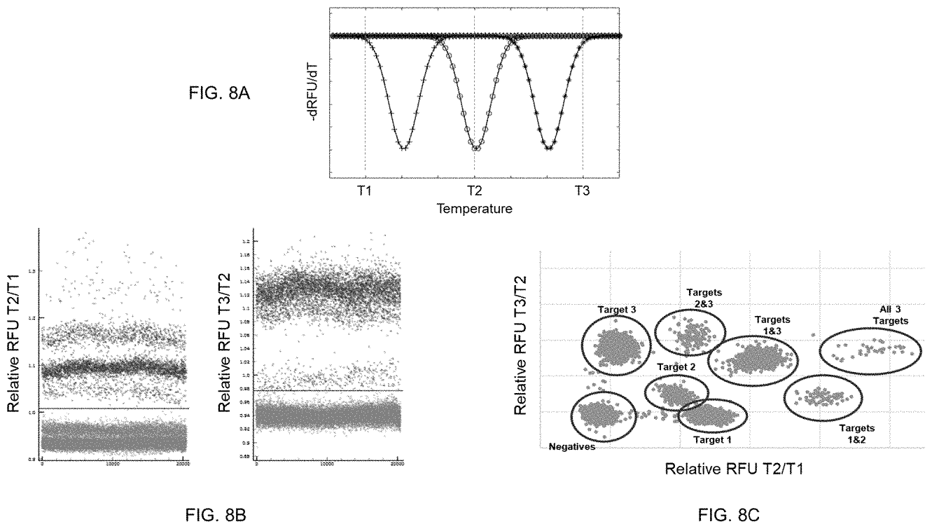

Ratios were calculated for fluorescence signals measured at successive temperatures: T2/T1 and T3/T2 and plotted on a 1D amplitude plot as shown in FIG. 8A and FIG. 8B respectively. The target duplex formed from probe JT435 has a Tm of 65° C. and is thus expected to show maximum change in fluorescence (ratio>1) between T1 and T2. Similarly, the target duplex formed from probe JB0043 has a Tm of 85° C. and is expected to show a maximum change in fluorescence (ratio>1) between T3 and T2. The target duplex formed from probe JT0410 has a Tm of 75° C. and is thus expected to show a change in fluorescence (ratio>1) between both T1 and T2 as well as between T3 and T2. As can be seen from FIG. 8A and FIG. 8B, multiple populations having ratios of different amplitudes can be seen in the 1D amplitude plots, but it is difficult to identify the expected 8 different target occupancy subsets of partitions with sufficient certainty to permit accurate quantitation. However, when the calculated ratios were subsequently plotted against each other as shown in FIG. 8C, the 8 different occupancy subsets are represented as 8 discernable clusters, permitting accurate quantitation using Poisson statistics without having to ignore multiple occupancy partitions and without having to determine a Tm or melt profile for partitions.

1-12. (canceled)

13. A method of distinguishing subsets of partitions in a multiplexed digital assay comprising a plurality of different target nucleic acids and a plurality of different probes, each different probe being specific for a different target nucleic acid and having a unique, predetermined Tm in the presence of its target nucleic acid, and wherein the plurality of different probes are labeled with the same reporter, the method comprising the steps of, for each partition:
a) detecting signals from the reporters of the plurality of different probes at three or more different predetermined temperatures;
b) calculating at least two relational values between signals measured at the three or more different predetermined temperatures;
c) plotting the at least two relational values against each other to distinguish subsets of partitions containing different combinations of the plurality of different target nucleic acids.

14. (canceled)

15. The method of claim 13, wherein each probe forms a duplex in the presence of its specific target nucleic acid and the reporter emits signal of different intensity in the duplex conformation than in single stranded conformation.

16. The method of claim 13, wherein prior to detecting, the partitions are subjected to an amplification reaction which includes the following steps:
e) hybridizing the plurality of different probes to their specific target nucleic acids if present;
f) cleaving the hybridized probes to form truncated probes;
g) hybridizing the truncated probes to respective capture sequences; and
h) extending the hybridized truncated probes to form duplexes having predetermined Tms unique to each different probe.

17-18. (canceled)

19. The method of claim 13, wherein the at least two relational values are calculated from signals detected at successive predetermined temperatures.

20. (canceled)

21. The method of claim 13, wherein there is a difference of at least five degrees C. between each of the three or more different temperatures at which signals are detected.

22-24. (canceled)

25. A method of quantifying a plurality of different target nucleic acids amplified in a digital assay in the presence of a plurality of different probes, each different probe being specific for one of the plurality of different target nucleic acids and distinguishable from other different probes by having a unique Tm, wherein signals from different probes are collected from a plurality of partitions and comprise the same signal-generating reporter, the method comprising the steps of:
a) performing a melt analysis to detect signals from the plurality of different probes in each partition at three or more different predetermined temperatures;
b) calculating at least two relational values for the signals detected at the three or more different predetermined temperatures for each partition;
c) plotting the at least two relational values against each other to identify subsets of partitions containing the same combinations of different target nucleic acids; and
d) quantifying the plurality of different target nucleic acids.

26. The method of claim 25, wherein calculating the at least two relational values comprises calculating ratios of signals detected at the three or more different predetermined temperatures.

27. The method of claim 25, wherein calculating at the least two relational values comprises calculating differences between signals detected at the three or more different predetermined temperatures.

28. The method of claim 25, wherein the at least two relational values are calculated from signals detected at successive predetermined temperatures.

29. The method of claim 25, wherein there is a difference of at least three degrees C. between each of the three or more different temperatures at which signals are detected.

30-31. (canceled)

32. The method of claim 25, wherein the method does not include determining the Tms of the plurality of different probes from the detected signals.

33. The method of claim 25, wherein the method does not include plotting a melt curve for each of the plurality of different probes from the detected signals.

34-35. (canceled)

36. A method for multiplexed detection in a digital PCR (dPCR) assay, the method comprising:
a) amplifying by a dPCR procedure one or more of a plurality of different target nucleic acids in a sample distributed across a plurality of partitions, wherein the dPCR procedure utilizes a plurality of different signal generating probes, each different signal generating probe being specific for one of the plurality of different target nucleic acids that may be present in the sample and being distinguishable in the presence of its specific target nucleic acid by having a unique melting temperature (Tm) relative to other different signal generating probes in the dPCR procedure;
b) detecting signals from the different probes in the plurality of partitions at three or more different predetermined temperatures;
c) calculating at least two relational values for signals measured at the three or more different predetermined temperatures for each of the plurality of partitions; and
d) plotting the at least two relational values against each other to determine which, if any, of the plurality of target nucleic acids are present in each of the plurality of partitions.

37. The method of claim 36, wherein calculating the at least two relational values comprises calculating ratios of signals detected at the three or more different predetermined temperatures.

38. The method of claim 36, wherein calculating the at least two relational values comprises calculating differences between signals measured at the three or more different predetermined temperatures.

39. The method of claim 36, wherein the at least two relational values are calculated from signals measured at successive predetermined temperatures.

40. The method of claim 36 wherein all different probes are labeled with the same signal generating reporter.

41. (canceled)

42. The method of claim 36, wherein there is a difference of at least five degrees C. between each of the three or more temperatures at which signals are detected.

43. (canceled)

44. The method of claim 36, wherein the method does not include determining the Tms of the plurality of different probes from the detected signals.

45. (canceled)

46. The method of claim 36, wherein each different probe forms a duplex in the presence of its specific target nucleic acid and emits signal of different intensity in the duplex conformation than in single stranded conformation.

47-85. (canceled)
